(19) United States
(12) Patent Application Publication
(10) Pub. No.: US 2021/0349093 A1
(43) Pub. Date: Nov. 11, 2021

(54) METHOD OF DEVELOPING VACCINES

(71) Applicant: The Board of Trustees of Western Michigan University, Kalamazoo, MI (US)

(72) Inventors: Robert Allen Makin, III, Kalamazoo, MI (US); Steven Michael Durbin, Kalamazoo, MI (US)

(73) Assignee: The Board of Trustees of Western Michigan University, Kalamazoo, MI (US)

(21) Appl. No.: 17/313,947

(22) Filed: May 6, 2021

Related U.S. Application Data
(60) Provisional application No. 63/021,435, filed on May 7, 2020.
(60) Provisional application No. 63/183,192, filed on May 3, 2021.

(51) Int. Cl. G01N 33/569

(52) U.S. Cl. CPC G01N 33/56983; G01N 2800/26; G01N 2333/11

(57) ABSTRACT

Immunogenic potential of a virus-like particle (VLP) for use in developing a vaccine is predicted by comparing order parameters of the VLP and a target virus. The method may include determining a numerical value of an order parameter and relative composition of viral coat proteins of a target virion (virus). A numerical value of an order parameter and relative composition of the surface proteins of a VLP is also determined. The numerical value of the order parameter and relative composition of the viral coat proteins of the target virion (virus) are compared to the numerical value of the order parameter and relative composition of the VLP to determine if the VLP satisfies pre-defined matching criteria indicative of high immunogenic potential and corresponding vaccine efficacy.

### CROSS-REFERENCE TO RELATED APPLICATION

This application claims the benefit under 35 U.S.C. § 119(e) to U.S. Provisional Patent Application No. 63/021,435, filed May 7, 2020, entitled “METHOD OF DEVELOPING VACCINES,” and U.S. Provisional Patent Application No. 63/183,192, filed May 3, 2021, entitled “QUANTITATIVE DISORDER ANALYSIS OF PHYSICAL SYSTEMS ACROSS LENGTH SCALES,” which are incorporated herein by reference in their entirety.

### BACKGROUND OF THE INVENTION

Viral vaccines may utilize active ingredients or components such as attenuated viral strains or virus-like particles (VLPs) to trigger an immune response. Bacterial vaccines may comprise toxoid, subunit, conjugate, inactivated, or live vaccines. VLPs are multiprotein structures that mimic the organization and conformation of viruses. VLPs may comprise the active component of effective vaccines, and may induce both innate and adaptive immune responses. Existing approaches to viral vaccine design may include developing attenuated viral strains or replicating select proteins to create a virus-like particle (VLP) which can trigger an appropriate immune response but are non-infectious. Understanding variations between viruses and vaccine strains therefore tends to focus on differences between proteins, which can be characterized through genetic analysis and in the context of structural motifs.

### BRIEF SUMMARY OF THE INVENTION

One aspect of the present disclosure is a method of developing a vaccine to treat or prevent diseases caused by viral or bacterial pathogens. The method includes predicting the immunogenic potential or immunogenicity of a VLP or other active vaccine component. The method may include determining a numerical value of an order parameter (S) (as used herein, “order parameter” may also generally refer to order parameter squared (S²) or other measure of order) in conjunction with the relative composition of viral coat proteins of a target virion (virus), or, more broadly, the relative composition of other active vaccine components. The method may further include determining a numerical value of an order parameter (S) and the relative composition of the surface proteins of a virus-like particle (VLP). The numerical value of the order parameter (and, optionally, relative composition of the viral coat proteins) of the target virion (virus) are compared to the numerical value of the order parameter (and, optionally, relative composition of the VLP) to determine if the VLP satisfies pre-defined matching criteria indicating that the VLP has sufficient immunogenic potential. Differences between the order parameters of the target virion/virus and the VLP may be utilized to determine if the VLP has sufficient predicted immunogenic potential. In general, smaller differences between the order parameters of the target virion (virus) and the VLP indicate that a VLP has higher immunogenic potential.

This approach may also be applied to develop vaccines to prevent or treat diseases caused by bacterial infections. In general, the order parameter of a potential active component may be compared to the order parameter of a target pathogen (bacteria), and predefined matching criteria can be utilized to determine if the potential active component satisfies pre-defined matching criteria indicating sufficient immunogenic potential.

The method may optionally include determining numerical values of the order parameter for a plurality of VLPs (or other active component) having non-identical structural motifs. One or more VLPs may be selected for a vaccine based, at least in part, on a degree to which the numerical values of the order parameters of the VLPs match the numerical value of the order parameter of the target virion (virus).

The method optionally includes determining a set of basis motifs (which may consist of a single or multiple motifs) of the viral coat proteins of the target virion (virus), wherein the base motif set comprises the minimum number of motifs to describe the capsid having a numerical value of the order parameter equal to one for the composition of the viral coat proteins of the target virion (virus).

The numerical value of the order parameter of the viron or the VLP may be one, zero, or a numerical value in between, depending on the composition of the viral coat proteins of the target virion (virus).

Another aspect of the present disclosure is a method of selecting a virus-like particle (VLP) or other active component for a vaccine. The method includes determining an order parameter of a target virion (virus), wherein the order parameter corresponds to a motif composition of the virion (virus). The method further includes determining an order parameter of one or more VLPs corresponding to motif compositions of the VLPs. At least one VLP is selected from the one or more VLPs by matching the motif composition of the VLP to the motif composition of the target virion (virus) utilizing the order parameters of the target virion (virus) and the VLPs. This approach may also be utilized to select an active component for vaccines to prevent or treat bacterial infections. In general, the order parameter of a potential active component can be compared to the order parameter of a target pathogen (bacteria) to determine a difference in order parameters. Potential active components having relatively small differences in order parameter compared to the target pathogen (e.g., bacteria) may be selected for further testing and evaluation.

Another aspect of the present disclosure is a method of controlling or adjusting the order parameter of a virus and/or a VLP by adjusting or varying the pH and/or incubation temperature of the virus and/or VLP.

### BRIEF DESCRIPTION OF THE DRAWINGS

FIG. 1A is a partially schematic image/illustration of a surface protein motif of a henipavirus particle showing a six-fold motif;

FIG. 1B is a partially schematic image/illustration of a beta-coronavirus particle including a spike protein, hemagglutinin-esterase protein, membrane protein, and an envelope protein;

FIG. 1C is a partially schematic image/illustration of a surface protein motif of an influenza virus particle;

FIG. 2A is a graph showing the percentage occurrence of different structural motifs at S=0 as a function of composition for an α3β4 base motif along with the complete set of variations for a viral coat protein;

FIG. 2B is a graph showing the percentage occurrence of different structural motifs at S=0 as a function of composition for a representative selection of single-viral coat protein dominated motifs;

FIG. 3 is a chart comparing order parameter (plotted as S²) for selected viruses and their corresponding VLP;

FIG. 3A is a graph showing vaccine efficacy verses differences in order parameter (S²) of pathogens and associated vaccines;

FIG. 4 is a graph showing the square of the order parameter as a function of incubation temperature for the Dengue fever virus and the influenza virus, including an inset showing the square of the order parameter (S²) as a function of the pH environment of the Dengue fever virus;

FIG. 5A is an Ising model analysis applied to virus-based diseases showing case fatality rate (also referred to as case fatality ratio) as a function of the pathogen order parameter (S²) for three viral strains as determined by analysis of published electronic microscopy images;

FIG. 5B is an Ising model analysis showing S² for health and disease-causing mitochondria;

FIG. 6 is a flowchart showing a procedure for calculating S² from a Raman spectrum;

FIG. 6A is a graph showing Raman spectroscopy data of an H1N1 VLP, along with the fitted ordered and disordered phase peaks; and

FIG. 6B is a graph showing the pixel intensity histogram of a TEM image of an H1N1 VLP.

### DETAILED DESCRIPTION

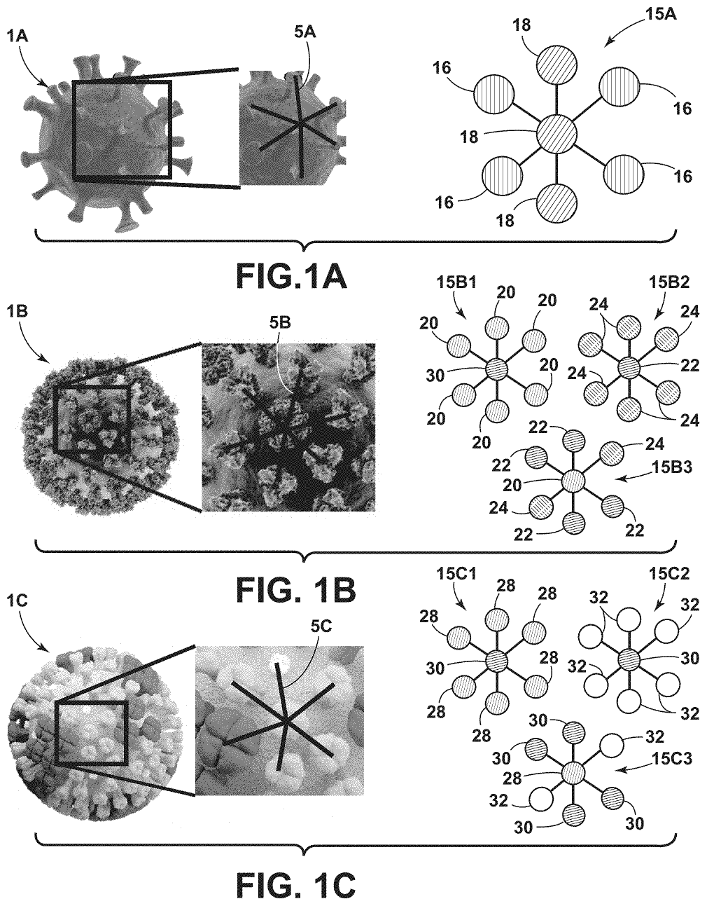

For purposes of description herein the terms “upper,” “lower,” “right,” “left,” “rear,” “front,” “vertical,” “horizontal,” and derivatives thereof shall relate to the disclosure as oriented in FIG. 1. However, it is to be understood that the invention may assume various alternative orientations and step sequences, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The present application is related to U.S. Provisional Patent Application No. 62/895,678, filed Sep. 4, 2019, and U.S. patent application Ser. No. 17/011,648, filed Sep. 3, 2020. The entire contents of each are incorporated herein by reference.

It is possible to experimentally quantify the degree of disorder and physical systems using a metric such as the Bragg-Williams order parameter (S). For a perfectly ordered system S=1. For a system with complete disorder S=0. Partially ordered systems exhibit a value of S between 0 and 1. As discussed in more detail below, one aspect of the present disclosure is a process for extracting order parameter S (or order parameter squared (S²)) from Raman spectra data.

In general, for a particular system (e.g., ZnSnN₂ or H1N1), constituent components are arranged adjacent to one another (zinc, tin, and nitrogen atoms, or hemagglutinin and neuraminidase proteins). The motifs describe the nearest neighbor environment. In the case of H1N1, the fully ordered case can be described with a single basis motif. However, as constituents are added, as is the case for coronaviruses, more than one motif is required to describe the fully ordered environment. As used herein, the term “basis motif set” could include a single motif. It will be understood that a motif (by itself) does not, in general, have an order parameter. Rather, the number of all of the different types of motifs (for some values of S, there will be zero numbers of some motifs) dictates the value of the order parameter S of a physical specimen for a given composition.

One aspect of the present disclosure is a method of developing vaccines that may include matching a motif composition of a VLP to a motif composition of a target virion (virus) utilizing order parameters of VLPs and viruses. Differences in the numerical magnitudes of order parameters of VLPs and viruses may be used to predict the immunogenic potential of a VLP. More generally, differences in the order parameters of active vaccine components and a corresponding target pathogen (e.g., virus or bacteria) may be used to predict the immunogenic potential of an active vaccine component.

As noted above, existing viral vaccine design may include developing attenuated viral strains or replicating select proteins to create a virus-like particle (VLP) which can trigger an appropriate immune response. Understanding variations between viruses and vaccine strains therefore tends to focus on differences between proteins, which can be characterized through genetic analysis and in the context of structural motifs. Although this approach provides information about the functioning and/or the interactions of the proteins, it does not always yield an early-stage pathway towards predicting the immunogenic potential of a VLP and efficacy of a vaccine. Thus, large-scale clinical trials may be required to obtain critical information. There is a need for earlier indications of whether a vaccine has the necessary characteristics, in both development and manufacturing stages.

The present disclosure demonstrates that it is possible to draw direct numerical correlations between virus particles and effective VLP-derived vaccines through extraction of a Bragg-Williams type order parameter from (for example) electron microscopy images. As discussed in more detail below, the type and occurrence of structural motifs within the arrangement of surface proteins may be determined by the numerical value of the order parameter as a measure of disorder. Application of an Ising model reveals that there is a clear relationship between case fatality rate and order parameter for distinct virus families. Additionally, the methodology disclosed herein has been applied to published results of Dengue and influenza virus particles. This demonstrates that temperature and pH during incubation may be controlled (adjusted) to fine-tune the order parameter of VLP-based vaccines to match the order parameter of the corresponding virus. The results discussed herein demonstrate the utility of being able to quantify the degree of disorder which characterizes the surface proteins of virus particles.

The present disclosure demonstrates that a previously “hidden” (unknown) variable identifies a characteristic of effective vaccines derived from virus-like particles (VLPs). An underlying concept relates to what are known as structural motifs, a familiar term in biology, especially with regard to proteins and understanding behavioral variation. The present disclosure shows that the notion of a structural motif can be extended to configurations of proteins on the surface of a virus particle. This aspect of the present disclosure extends the concept beyond variations within the protein structure itself, which is the conventional use of the term. The advantages of doing so can be significant because the disorder and relative composition characterize surface configurations that are preferably preserved in vaccines developed from corresponding virus-like particles. Further, the quantifiable degree of disorder can be used in the context of an Ising model to predict case fatality rate of a new variant to a known virus, and to identify process parameters (e.g., pH and temperature, to be monitored/measured/controlled/adjusted) during incubation of potential vaccine components.

FIGS. 1A, 1B, and 1C illustrate surface protein motifs of several known virus particles. A common structural feature of viruses is the outer-most layer of proteins, which play a role in activities such as binding to host cells and injecting viral genetic material into those cells. The outer proteins (referred to as “viral coat proteins”) also play an important role in the immune system response, since antibodies may bind to these proteins in order to signal the immune system to attack the virus. It is possible to view the configuration of these outer-most proteins from a structural motif perspective, as they are typically six-fold coordinated (although other coordination numbers are possible), with a central protein surrounded by six other proteins that form the basis of the hexagonal-close packed lattice structure.

There are numerous possible permutations of six-fold motifs. In the case of a virus with two major viral coat proteins (which, to avoid loss of generality, will be referred to herein as α and β) as depicted in FIG. 1A, there are 20 distinct six-fold motifs possible. The number of each motif occurring in a particular specimen may be determined by the viral coat protein composition and by the degree of disorder characterizing the virus surface, the latter of which can be quantified through a Bragg-Williams type order parameter S (also referred to herein as “long range order parameter”) (see, e.g., Bragg, W. L. and Williams, E. J., “The effect of thermal agitation on atomic arrangement in alloys,” Proceedings of the Royal Society of London, Series A, Containing Papers of a Mathematical and Physical Character, Vol. 145, pp. 699-730, 1934; and Cullity, B. D., “Elements of x-ray diffraction,” Addison-Wesley Publishing Company, Inc., Reading, Mass., 1978). A fully ordered surface, corresponding to a unity value of S, consists of only a single structural (basis) motif, illustrated in FIG. 1A for the case of six-fold coordinated viruses with two major viral coat proteins. For the cases of more than two types of viral coat proteins, a set of basis motifs is required to describe the fully ordered surface, as is shown in FIGS. 1B and 1C for the cases of six-fold coordinated viruses with three and four viral coat proteins. In contrast, a completely disordered protein configuration corresponds to a value of S=0, and can have a large set of structural motifs representing variations of the set of basis motifs.

With reference to FIG. 1A, a henipavirus virus particle 1A includes a six-fold basis motif 5A. With reference to the schematic representation 15A of the six-fold base motif 5A, the basis motif includes four fusion proteins 16 and three glycoproteins 18.

With reference to FIG. 1B, a beta-coronavirus virus particle 1B may include a set of three basis motifs 15B1, 15B2, and 15B3 which are shown schematically. The specimen specific motifs may include spike proteins 20, membrane proteins 22, and envelope proteins 24.

With reference to FIG. 1C, an influenza virus particle 1C includes a six-fold basis motif 5C. Schematic representations of three basis motifs 15C1, 15C2, and 15C3 include M2 ion channel proteins 28, hemagglutinin glycoprotein 30, and neuraminidase glycoprotein 32.

It will be understood that the present disclosure is not limited to the specific viral particles of FIGS. 1A, 1B, and 1C.

In general, there are at least two significant factors that determine the occurrence probability of the different motifs. A first factor is the ordering of the viral coat proteins on the outer surface of the virion. As discussed below, a second factor that determines the occurrence probability of different motifs is the relative amounts of the different viral coat proteins.

To proceed with developing a quantifiable measure of the degree of ordering, we first define the perfectly ordered (S=1) case. This is accomplished by selecting the base motif—the structural motif which contains equal numbers of both viral coat proteins, identified in FIG. 1B. For this case, the outer viral coat proteins of the motif only contribute ½ to the viral coat protein count since they are shared by neighboring motifs. Using the base motif as a reference, the degree of ordering can be quantified through the Bragg-Williams order parameter S, where:

S=r_{α}+r_{β}−1 (1)

Where, r_{α} is the fraction of α viral coat proteins on α-sites, and r_{β} is the percentage of β viral coat proteins on β-sites, respectively (from the perspective of the reference (base) motif). The approach may be extended for situations involving more than two distinct viral coat proteins. In material systems such as binary metal alloys or semiconductors, the order parameter may be measured utilizing techniques such as x-ray diffraction. Recently, the determination of S has been extended to include techniques such as Raman spectroscopy (see, e.g., Makin, R. A. et al., “Alloy-Free Band Gap Tuning across the Visible Spectrum,” Phys. Rev. Lett., 122, 256403, 2019) and electron microscopy images (see, e.g., Makin, R. A. et al., “Revisiting semiconductor band gaps through structural motifs: An Ising model perspective,” Phys. Rev. B 102, 115202, Sep. 8, 2020), the latter of which has been used to calculate the order parameter of virions from transmission electron microscopy (TEM) images.

As noted above, another significant factor that determines the occurrence probability of different motifs is the relative amount of the different viral coat proteins. For the case of two viral coat proteins, the fractional composition may be defined as:

$$x = \frac{N α}{( {N}_{α} + {N}_{β} )} \qquad ( 2 )$$

Where x is the fractional composition and N_{α} and N_{β} represent the total number of each protein on the particle, respectively. N_{α} and N_{β} can be measured through techniques such as sodium dodecyl sulfate-polyacrylamide gel electro-phoresis (see, e.g., Gels, T. et al., “Tricine-sodium dodecyl sulfate-polyacrylamide gel electrophoresis for the separation of proteins in the range from 1 to 100 kDa,” Anal. Biochem., 166, pp. 368-379, 1987). The percentage of different motifs that will occur on the surface of the virion can be calculated based on the fractional composition x and the order parameter S.

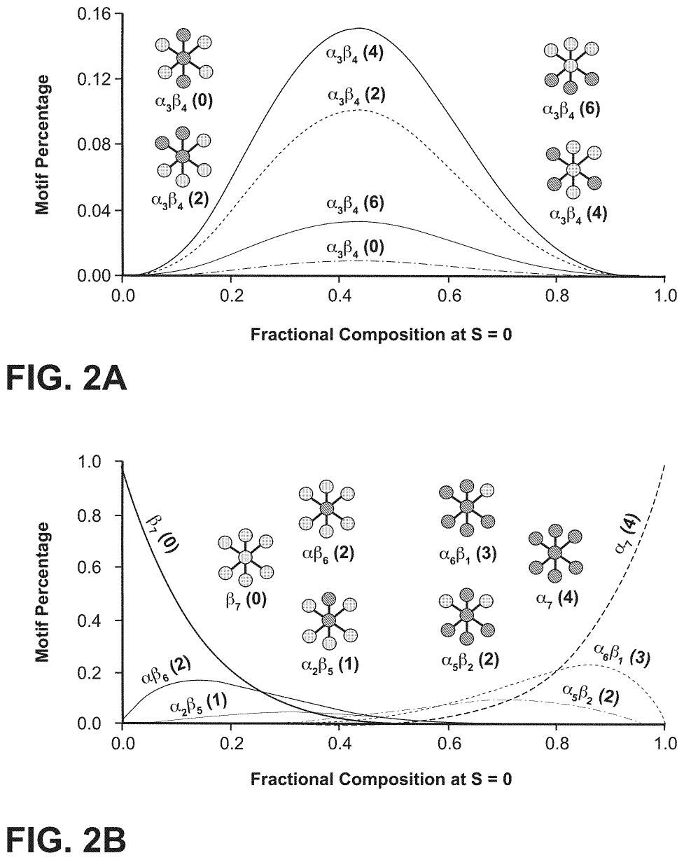

FIGS. 2A and 2B show the motif percentages for a fully disordered surface over the full range of possible compositions of a 2-viral coat protein, 6-fold coordinated motif. As illustrated in FIG. 2A, the basis set of motifs is much more likely to form at balanced compositions, i.e., near x=0.5. At the extreme values of x, the motifs dominated by a single viral coat protein (β7 and α7 motifs) become much more likely to form, and specific types of single viral coat protein dominated motifs (such as the α2β5 or α6β1 motifs) become more likely with shifts towards the extreme ranges of the fractional composition x, as can be seen in FIG. 2B. Depending on the fractional composition x, there may be physically inaccessible values of 5; the maximum S value for x≤0.5 is 2x and 2(1−x) for x≥0.5.

Viral coat proteins play a defining role in the interactions between viruses, host cells, and antibodies, and the viral coat protein structural motifs play a significant role in these interactions. Thus, the present disclosure involves matching the motif composition of the intended virus when developing vaccines starting with VLPs. However, counting the motifs of a given virus or VLP may be impractical. Thus, utilizing the correlation between the numerical occurrence of specific motifs and the order parameter for known viral coat protein composition provides a viable alternative.

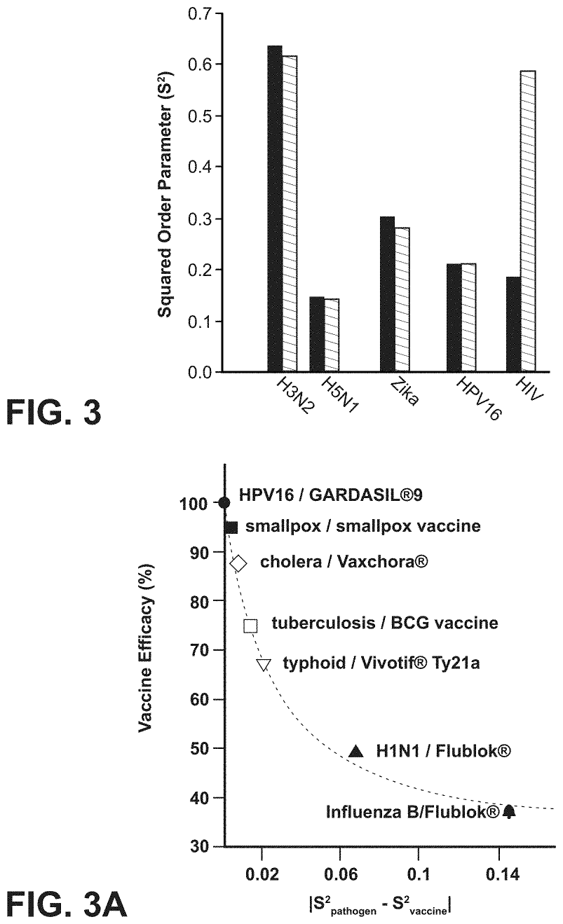

FIG. 3 compares order parameter values (expressed as the square of the order parameter) (i.e., S²) extracted from TEM images for five distinct viruses (H3N2, H5N1, Zika, HPV16, and HIV) to the corresponding experimentally determined order parameters for VLPs used to create vaccines. The VLPs for the two influenza viruses and for the Zika virus have order parameters very close to their corresponding viruses, and showed promising results for developing immune responses (i.e., high immunogenic potential). Conversely, for the example HIV vaccine of FIG. 3, the VLP has an order parameter that is much higher than that of the virus. A possible explanation for the difficulty in developing an effective HIV vaccine could be that current vaccines and even potential vaccines, such as yeast-based VLPs (the S² of one such yeast-based VLP is shown in FIG. 3), do not properly match the order parameter and composition of the viral coat surface of the HIV virus. This suggests that for a VLP to have sufficient immunogenic potential, whereby a vaccine based on the VLP is effective, the order parameter of any VLP used in the creation of the vaccine should closely match that of the corresponding virus.

Referring again to FIG. 3, differences between the numerical S² values of VLPs and the corresponding virus for effective vaccines is significantly less than 0.1. In particular, in the examples of FIG. 3, the differences in the order parameters is about 0.02 for H3N2 and Zika, and significantly less than 0.02 for H5N1 and HPV16. In contrast, the difference in order parameter (S²) for HIV and the example vaccine shown is greater than 0.4.

Similarly, as shown in FIG. 3A, smaller differences between squared order parameter (S²) of a pathogen (virus or bacteria) and squared order parameter (S²) of a vaccine to treat the pathogen are associated with increased vaccine efficacy. Thus, the order parameter can be utilized to predict immunogenic potential of active components for both viral and bacterial vaccines.

In general, the order parameters (e.g., S²) of several VLPs (active components) may be determined and compared to the order parameter of a target virus (or bacteria), and one or more VLPs having the greatest immunogenic potential (smallest difference in order parameter) may be selected for further development. Also, VLP “pass/fail” selection criteria involving differences in order parameter may be utilized to include or exclude VLPs for further development. For example, if a selection criteria of 0.1 is used, all VLPs having a difference in the square of the order parameter (S²) greater than 0.1 relative to the square of order parameter (S²) of the target virus could be excluded from further consideration. Alternatively, the selection criteria could be more stringent (e.g., a difference in S² of no more than 0.02) if warranted by the circumstances. As noted above, this approach may also be utilized to identify active components to be used in vaccines to treat or prevent diseases caused by bacterial infection.

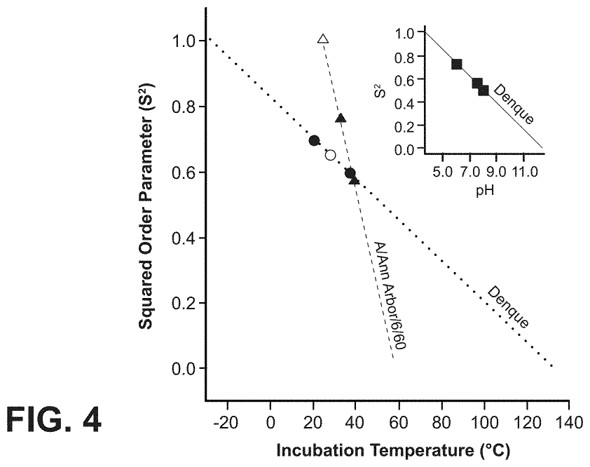

When developing VLPs (or any vaccine), the composition of the viral coat proteins can be controlled genetically. However, the degree of disorder which characterizes the viral coat protein surface of a virus is influenced by conditions under which the virion matures. Examples of such conditions are the temperature and/or the pH of the growth environment. This is observed in the plot of FIG. 4, which shows the variation of the squared order parameter (S²) in Dengue and influenza viruses with varying incubation temperatures in a neutral pH environment. The linear variation of the square of the order parameter (S²) with temperature may be indicative of a Landau second-order phase transition present in ordered-disordered systems. Additionally, the inset (FIG. 4) shows the influence of pH on the square of the order parameter (S²) of the Dengue virus, which follows a similar linear trend as the temperature.

Further insight into viruses can be achieved by applying the classic Ising model to the viral coat proteins, where the α viral coat protein is assigned a spin “up” and the β viral coat protein is assigned a spin “down.” This is a special case of the more general multi-spin Potts model. Such an approach, in conjunction with cluster expansion theory, makes it possible to cast a physical or system property P (provided it is dominated by the action of both viral coat proteins) as:

P(S, x)=[P(S=1, 0.5)−P(S=0, x)]S²+P(S=0, x) (3)

The Ising model equation therefore predicts a linear correlation of the property P to the square of the order parameter (S²).

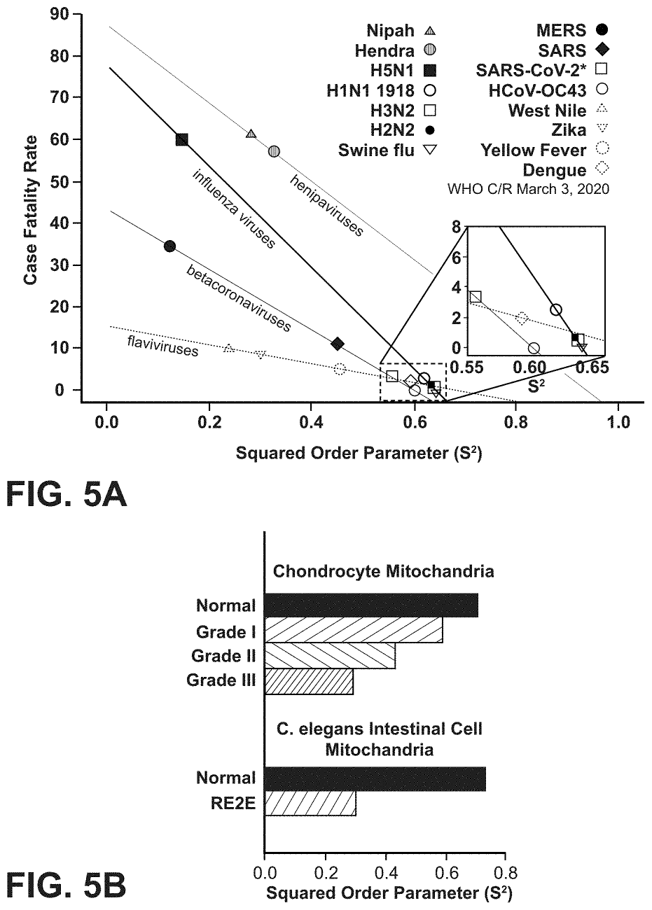

Applying the Ising model to four different virus families (henipaviruses, flaviviruses, influenza viruses, and coronaviruses), with the case fatality rate of the virus as the system property, yields remarkable agreement as illustrated in FIG. 5A. It suggests, among other things, that such an approach enables a prediction of the severity of any new virus provided that other members of the same family are well documented. Although not wishing to be bound by theory, a possible explanation for the linear trends observed in FIG. 5A can be found from within a motif perspective. In particular, it can be observed that for all four families, as the disorder increases, the fatality rate also increases. Increasing disorder may be correlated with more single-type viral coat protein motifs, for example, more hemagglutinin-heavy motifs in the case of influenza, envelope-heavy motifs in flaviviruses, and spike-heavy motifs in coronaviruses. These viral coat proteins bind to the host cells, so a higher number of such motifs may translate into a higher probability of the virions successfully attaching and subsequently infecting host cells, and a corresponding higher case fatality rate seen in each family with decreasing S² values.

Also, although all of the available experimental data points on the Ising model plot have a positive value for case fatality rate, extrapolating the lines to the fully ordered (S=1) case yields negative values. Far from being unphysical, negative values may be explained by considering that the opposite to fatality would be a measure of symbiosis enabled by the virus. An example that supports this interpretation comes from studies of mitochondria (in which the equivalent to viral coat proteins would be the two types of porins on its outer-surface) and corresponding diseases. As shown in FIG. 5B, healthy mitochondria that exist in cells have very high S² values, while mitochondria that are responsible for diseases have a lower S² value.

In general, the process for evaluating a new viral (or bacterial) pathogen generally includes determining ratio of capsid proteins using a technique such as sodium dodecyl sulfate-polyacrylamide gel electrophoresis (an alternative but functional equivalent route is required for the case of a bacterial pathogen). This fixes the fractional composition “x” (FIGS. 2A and 2B), and determines the “line” in an Ising model plot. In order to have a high-degree of efficacy, “x” must be matched between a vaccine and a pathogen as well as order parameter S.

The present disclosure provides a methodology for viewing viruses through the lens of viral coat motifs. Specifically, disorder and relative fractional composition of viral coat proteins determine the range of viral coat protein structural motifs present on viruses and VLPs used in vaccines. The present disclosure includes a method for quantizing the degree of disorder through an order parameter, 5, which can be measured using electron microscopy images. Additionally, combining a quantitative measure of disorder with an Ising model potentially allows for deeper insights into the root cause for virus properties in a population, as well as guidance in terms of predicting immunogenic potential or immunogenicity and achieving the characteristics needed for vaccines.

### Methods

The order parameter for lattice structures can be measured using a variety of experimental techniques, such as x-ray diffraction, Raman spectroscopy or electron diffraction. The order parameter of a sample may also be calculated from transmission electron microscopy (TEM) images. In such images, the pixel intensity is less in disordered regions than in ordered regions. This stems from the fact that electrons are incoherently scattered by disordered stacks of atoms as opposed to the coherent diffraction that occurs from well-ordered stacks of atoms. The S² value (i.e., the square of the order parameter) of a sample is, in general, equal to the percentage of area corresponding to bright regions. The bright and dark areas corresponding to the ordered and disordered regions can be more easily determined and measured by thresholding the image near the average pixel intensity of the bright regions.

The following discussion concerns equivalence between the methods of calculating S² from TEM and Raman spectroscopy, specifically surface-enhanced Raman spectroscopy (SERS). The order parameter for lattice structures can be accurately measured using a variety of experimental techniques, such as x-ray diffraction, Raman spectroscopy, and electron diffraction (see, e.g., Makin, R.A. et al., “Alloy-Free Band Gap Tuning across the Visible Spectrum,” Phys. Rev. Lett., 122, 256403, 2019).

Table S1 is a comparison of order parameter (S) values extracted from transmission electron microscopy (TEM) and surface-enhanced Raman spectroscopy (SERS) provided as S² for example influenza virus.

TABLE S1

| VLP | S² from TEM | S² from SERS |
|---|---|---|
| H1N1 | 0.6786 | 0.6783 |
| H3N2 | 0.7271 | 0.7273 |
| H5N1 | 0.7520 | 0.7521 |

Table S1 provides evidence demonstrating the equivalence between transmission electron microscopy (TEM) and surface-enhanced Raman spectroscopy (SERS) with regards to measuring/determining order parameter (S²).

Extracting the Order Parameter from Raman Spectra

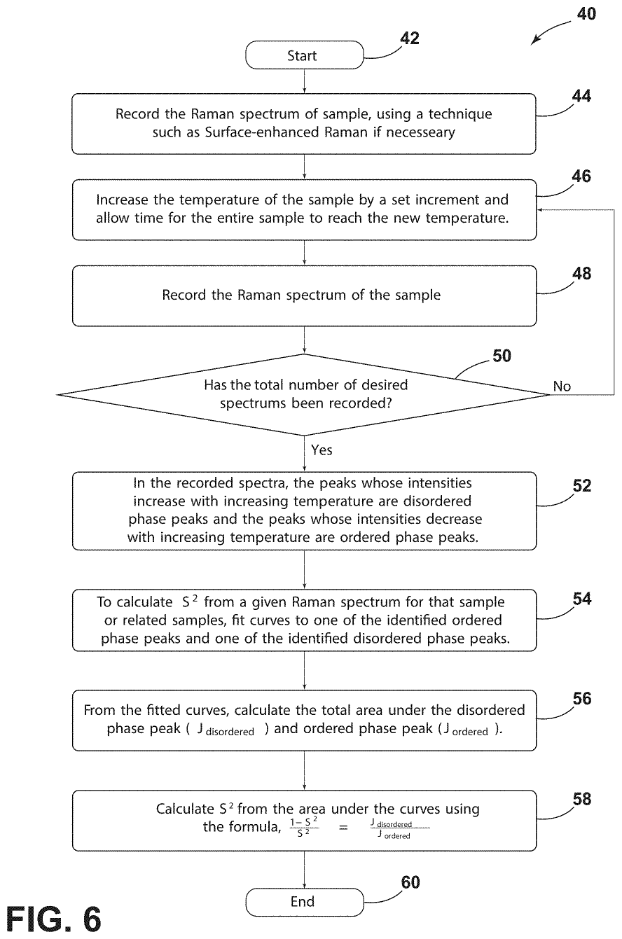

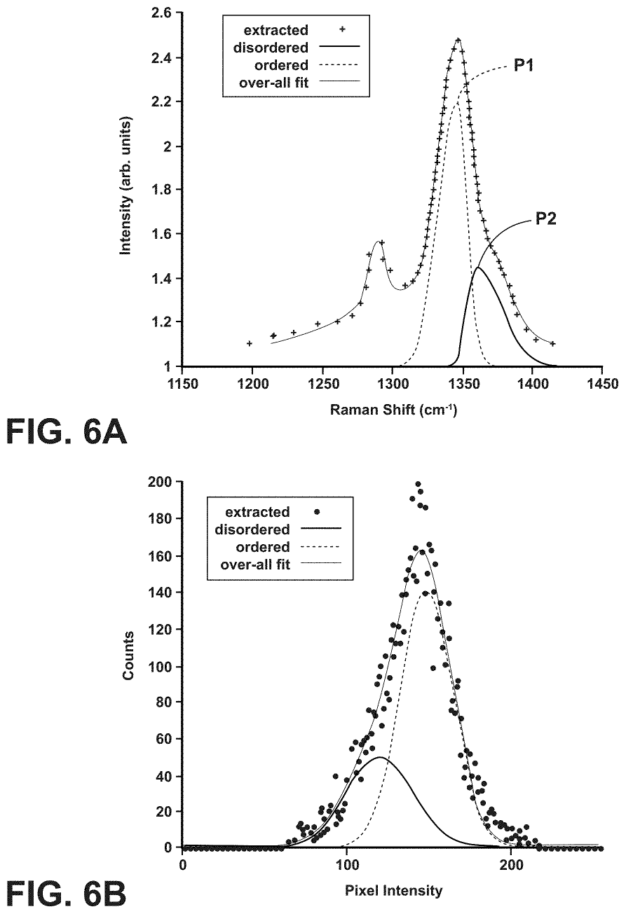

With reference to FIG. 6, a process 40 for calculating S² from Raman spectrum starts at 42, and the Raman spectrum of a sample is then recorded at 44 using a technique such as a surface-enhanced Raman if necessary. The temperature of the sample is increased by a set increment and time is allowed for the entire sample to reach the new temperature at step 46. The Raman spectrum in the sample is then recorded at step 48. As shown at step 50, the process repeats steps 46 and 48 unless the total number of the desired spectra have been recorded. Once the total number of desired spectra have been recorded, the process continues to step 52. At step 52, the recorded spectrum is evaluated to determine the disordered phase peaks and the ordered phase peaks. Specifically, the peaks having intensities that increase with increasing temperature are determined to be disordered, and peaks having intensities that decrease with increasing temperature are determined to be ordered phase peaks. Examples of intensity vs. Raman shift is shown in FIG. 6A and pixel counts vs. pixel intensity is shown in FIG. 6B.

At step 54, S² is calculated from a given Raman spectrum for that sample or related samples, and curves are fit to one of the identified ordered phase peaks and one of the identified disordered phase peaks.

At step 56, the total area under the disordered phase peak (J_{disordered}) and ordered phase peak (J_{ordered}) are calculated from the fitted curves. Finally, at step 58, S² can be calculated from the area under the curves using the formula:

$$\frac{1 - {S}^{2}}{{S}^{2}} = \frac{{J}_{d i s o r d e r e d}}{{J}_{o r d e r e d}} \qquad ( 4 )$$

The process 40 of FIG. 6 then ends at 60.

With reference to FIG. 6A, in the case of influenza VLPs, the peak P1 near 1340 cm⁻¹ corresponds to contributions from the ordered motif (which exhibits an S² intensity dependency) and the peak P2 near 1380 cm⁻¹ corresponds to contributions from the other motifs (which exhibit a 1−S² intensity dependency), and only appear for S<1, as shown in FIG. 1A for the H1N1 VLP. Calculating the area under each of the fitted peaks by integration gives P1 29.585 for the ordered peak and 14.029 for the disordered peak. The set of equations given in Makin, R. A. et al., “Alloy-Free Band Gap Tuning across the Visible Spectrum,” Phys. Rev. Lett., 122, 256403, 2019, can be rearranged to solve for S² given an ordered peak and disordered peak in the same sample:

$$( \frac{{J}_{S = 1}}{{J}_{S = 0}} ) = \frac{{S}^{2}}{1 - {S}^{2}} \qquad ( 5 )$$

Applying the data in Lin, Yu-Jen et al., “A Rapid and Sensitive Early Diagnosis of Influenza Virus Subtype via Surface Enhanced Raman Scattering,” Journal of Biosensors & Bioelectronics, Vol. 5:2, 2014, yields an S² value of 0.6783, as shown in Table S1.

Extracting the Order Parameter from Transmission Electron Microscopy

The S² value of a sample is equal to the percentage of sample image area corresponding to bright regions. The bright and dark areas corresponding to the ordered and disordered regions can be more easily detected and measured by thresholding the image near the average pixel intensity of the bright regions. In the TEM image from FIG. 3 of Lin, Yu-Jen et al., “A Rapid and Sensitive Early Diagnosis of Influenza Virus Subtype via Surface Enhanced Raman Scattering,” Journal of Biosensors & Bioelectronics, Vol. 5:2, 2014, the average bright area pixel value was approximately 140. Thresholding the image at a pixel value of 133, the percentage of area found to contain bright ordered pixels is 67.86. This yields an S² value of 0.6786, in good agreement with the S² value obtained from SERS, as shown in Table S1.

It is to be understood that variations and modifications can be made on the aforementioned disclosure without departing from the concepts of the present invention, and further it is to be understood that such concepts are intended to be covered by the following claims unless these claims by their language expressly state otherwise. Also, it will be understood that the term “order parameter” as used herein generally refers to the order parameter (S), the square of the order parameter (S²), and other quantitative measures, expressions, or descriptions of order. Furthermore, the concepts described herein may be utilized in connection with vaccines to treat humans and animals to prevent and/or treat diseases that are caused by viruses, bacteria, or virtually any other pathogen.

The invention claimed is:

1. A method of predicting the immunogenic potential of a virus-like particle (VLP) for a vaccine, the method comprising:
determining a numerical value of an order parameter (S or S²) of viral coat proteins of a target virion/virus;
determining a numerical value of an order parameter (S or S²) of a virus-like particle (VLP); and
comparing the numerical value of the order parameter (S or S²) of the viral coat proteins of the target virion/virus to the numerical value of the order parameter (S or S²) of the VLP to determine if the VLP satisfies predefined matching criteria indicative of sufficient immunogenic potential.

2. The method of claim 1, including:
determining numerical values of the order parameter (S or S²) for a plurality of VLPs having non-identical structural motifs; and
selecting one or more VLPs for a vaccine based, at least in part, on a degree to which the numerical values of the order parameters (S or S²) of the VLPs match the numerical value of the order parameter (S or S²) of the target virion/virus.

3. The method of claim 1, including:
determining a basis set of motifs of the viral coat proteins of the target virion/virus, wherein the basis motif set is the minimum number of structural motifs which describe the capsid proteins and their arrangement corresponding to the special case of a numerical value of the order parameter (S or S²) equal to one for the composition of the viral coat proteins of the target virion/virus.

4. The method of claim 3, including:
comparing the numerical value of the order parameter (S or S²) of a basis motif set to the numerical value of the order parameter (S or S²) of a VLP to determine if the VLP satisfies predefined matching criteria indicative of sufficient immunogenic potential.

5. The method of claim 1, wherein:
the predefined matching criteria comprises a difference between the numerical value of the order parameter (S or S²) of the target virion/virus and the numerical value of the order parameter (S or S²) of the VLP.

6. The method of claim 1, including:
adjusting the order parameter (S or S²) of a VLP by controlling at least one incubation condition selected from the group consisting of temperature and pH.

7. A method of evaluating virus-like particles (VLPs) for a vaccine, the method comprising:
determining an order parameter (S or S²) of a target virion/virus corresponding to a motif composition of the virion/virus;
determining an order parameter (S or S²) of a plurality of VLPs corresponding to motif compositions of the VLPs; and
utilizing the order parameters (S or S²) of the target virion/virus and the VLPs to determine which VLPs have motifs that are most closely matched to the motifs of the virion/virus for the same fractional composition of proteins.

8. The method of claim 7, including:
comparing the order parameters (S or S²) of the VLPs to the order parameter of the virion/virus.

9. The method of claim 8, including:
determining a difference between the order parameter (S or S²) of each VLP and the order parameter of the virion/virus.

10. The method of claim 9, including:
selecting the VLP having the smallest difference in order parameter (S or S²) for use in a vaccine.

11. The method of claim 7, including:
rejecting VLPs having motifs that are insufficiently matched to motifs of the target virion/virus utilizing predefined acceptance criteria.

12. The method of claim 11, wherein:
the predefined acceptance criteria comprises a numerical value representing a maximum difference in order parameters (S or S²) of the target virion/virus and the VLP.

13. A method of predicting immunogenic potential of a virus-like particle (VLP) to be used in a vaccine, the method comprising:
determining an order parameter (S or S²) of a viral coat protein of a selected virus;
determining an order parameter (S or S²) of the VLP;
comparing the order parameter (S or S²) of the viral coat protein to the order parameter (S or S²) of the VLP; and
utilizing predefined criteria to determine if the order parameter (S or S²) of the viral coat protein sufficiently matches the order parameter (S or S²) of the VLP to indicate that the VLP has sufficient predicted immunogenic potential for use in a vaccine designed to stimulate an immune response to the selected virus.

14. The method of claim 13, wherein:
the order parameter (S²) of the VLP is determined from at least one of Raman spectroscopy (SERS) data and electron microscopy (EM).

15. The method of claim 13, wherein:
the predefined criteria comprises a numerical value between zero and one;
determining the square of the order parameter (S²) of the viral coat protein of a selected virus;
determining the square of the order parameter (S²) of the VLP;
determining a numerical difference between the S² of the viral coat protein of a selected virus and the S² of the VLP;
comparing the numerical difference to the numerical value; and
determining that the VLP has sufficient immunogenic potential if the numerical difference is less than the numerical value.

16. The method of claim 13, wherein:
the numerical value is in the range of 0.0-0.1.

17. The method of claim 14, wherein:
the order parameter (S²) of the VLP is determined from VLP SERS data by:
1) identifying first and second intensity peaks corresponding to first and second Raman shifts, respectively, wherein the first Raman shift corresponds to an ordered region and the second Raman shift corresponds to a disordered region;
2) calculating a first area (J_{S=1}) under the first intensity peak and a second area J_{S=2}) under the second intensity peak; and
3) calculating S² of the VLP using an equation of the form:
$$( \frac{{J}_{S = 1}}{{J}_{S = 0}} ) = \frac{{S}^{2}}{1 - {S}^{2}}$$

18. The method of claim 14, wherein:
the order parameter (S²) of the VLP is determined from VLP TEM data comprising a sample image area having bright areas corresponding to ordered regions and dark areas corresponding to disordered regions by determining an average pixel intensity of the bright areas of the sample image area, thresholding the image near the average pixel intensity of the bright areas, and determining a ratio of the bright area within the threshold to the sample image area, wherein S² of the VLP is equal to the ratio.

19. A method of predicting the immunogenic potential of an active component for a vaccine, the method comprising:
determining a degree of order of a target pathogen;
determining a degree of order of an active component for a vaccine; and
comparing the degree of order of the target pathogen to the degree of order of the active component to determine if the active component satisfies predefined matching criteria indicative of sufficient immunogenic potential.

20. The method of claim 19, wherein:
the degree of order of the target pathogen comprises an order parameter;
the degree of order of the active component comprises an order parameter;
the pathogen comprises a virus or a bacteria, and including:
determining numerical values of the order parameter (S or S²) for a plurality of active components having non-identical structural motifs; and
selecting one or more active components for a vaccine based, at least in part, on a degree to which the numerical values of the order parameters (S or S²) of the active components match the numerical value of the order parameter (S or S²) of the target pathogen.
